(19) United States
(12) Patent Application Publication
(10) Pub. No.: US 2014/0275906 A1
(43) Pub. Date: Sep. 18, 2014

(54) GARMENT FOR A MEDICAL DEVICE

(71) Applicant: Steven Richard Hackenburg, Queensbury, NY (US)

(72) Inventor: Steven Richard Hackenburg, Queensbury, NY (US)

(21) Appl. No.: 13/832,062

(22) Filed: Mar. 15, 2013

(51) Int. Cl. A41D 13/12; A61B 5/00; A61M 5/142; A41D 1/00

(52) U.S. Cl. CPC A41D 13/1236; A41D 1/002; A61B 5/6804; A61M 5/142
USPC 600/365; 2/69; 2/109; 450/1; 604/151

(57) ABSTRACT

A garment for a medical device comprises a front panel; a back panel; at least one opening for a body part; at least one pocket; and at least one fastener. In some embodiments, the garment can be worn by the user of an insulin pump.

### BACKGROUND OF THE INVENTION

An insulin pump is a medical device used for the administration of insulin or the administration of insulin genes in the treatment of diabetes mellitus, also known as continuous subcutaneous insulin infusion therapy. The insulin pump can be used as an alternative to multiple daily injections of insulin by an insulin syringe or an insulin pen. The device typically includes: the pump, including controls, processing module, and batteries; a disposable reservoir for insulin, typically located inside the pump; and a disposable infusion set, including a cannula for subcutaneous insertion and a tubing system to interface the insulin reservoir to the cannula.

Users of the insulin pump report better quality of life compared to using other devices for administering insulin, however there are some disadvantages yet to be addressed, including discomfort and unwieldiness, for example during physical activity or sleep.

### SUMMARY

According to an aspect of the invention, a garment for a medical device comprises a front panel; a back panel; at least one opening for a body part; at least one pocket; and at least one fastener.

The garment can comprises a garment to be worn by a male or female user of a medical device selected from the group consisting of: an undergarment such as such as underwear, a pair of boxers, a pair of boxer-briefs, a thong, or a pair of boy-shorts; a shirt such as a long-sleeved, a short-sleeved, or a sleeveless shirt; a pair of shorts; a pair of pants; swimwear; athletic wear; a camisole; a brassiere; a sports brassiere; and a belt.

The garment can comprise a material selected from the group consisting of: cotton; lyocell; bamboo; viscose; elastane; polyester; nylon; polychloroprene; a waterproof material; and combinations thereof.

The garment can further comprise a waistband, and the at least one opening can comprise a pair of leg openings. The medical device can comprise a tubular structure selected from the group consisting of: a tube; a catheter; a wire; a cable; a cord; and combinations thereof, and the tubular structure can be secured to a surface of the waistband via the at least one fastener. For example, the garment can comprise at least five fasteners, where two of the at least five fasteners are positioned on the front panel, one of the at least five fasteners is positioned on the back panel, one of the at least five fasteners is positioned on a hip region, and one of the at least five fasteners is positioned on an opposite hip region.

The at least one opening can comprise a pair of arm openings. The medical device can comprise a tubular structure selected from the group consisting of: a tube; a catheter; a wire; a cable; a cord; and combinations thereof, and the tubular structure can be secured to a surface of the front panel and/or a surface of the back panel.

The at least one pocket can be configured to support a portion of the medical device. For example, the portion of the medical device can comprise a pump portion of an insulin pump.

The at least one pocket can be sized to support a portion of the medical device having dimensions of approximately 77 mm (length)×47 mm (width)×18 mm (height).

The at least one pocket can be configured to support up to approximately 16 ounces.

The at least one pocket can comprise a material having minimal stretching.

The at least one pocket can be positioned on the front panel and/or the back panel.

The at least one pocket can comprises a first pocket and a second pocket, where the first pocket can be positioned on a hip region and the second pocket can be positioned on the opposite hip region.

The at least one pocket can comprise a hook and loop fastener; at least one button; a zipper; and combinations thereof configured to close the at least one pocket.

The at least one fastener can be configured to secure a tubular structure of the medical device to the garment. The at least one fastener can be integral to the garment and/or the at least one fastener can be a separate component.

The at least one fastener can comprise a first end and a second end, where the first end comprises a closed portion and the second end comprises an opening configured to limit pinching, kinking or affecting the functionality of the tubular structure. The opening can be sized to allow a tubular structure having an outer diameter ranging from approximately 0.1 mm to 10 mm to pass therethrough.

The medical device can comprise an insulin pump; an infusion device; a blood sugar monitoring device; an insulin inhaler; or a defibrillator.

The at least one fastener can comprise a fastener selected from the group consisting of: a pin; a clip; a medical hose with a reinforcing wire; a loop; and combinations thereof.

According to another aspect of the invention, a garment for a medical device comprises a front panel; a back panel; a pair of leg openings; a waistband; at least one pocket configured to support a portion of the medical device; and at least one fastener configured to secure a tubular structure of the medical device to a surface of the waistband.

According to another aspect of the invention, a garment for a medical device comprises a front panel; a back panel; a pair of arm openings; at least one pocket configured to support a portion of the medical device; and at least one fastener configured to secure a tubular structure of the medical device to a surface of the front panel and/or a surface of the back panel.

The technology described herein, along with the attributes and attendant advantages thereof, will best be appreciated and understood in view of the following detailed description taken in conjunction with the accompanying drawings in which representative embodiments are described by way of example.

### BRIEF DESCRIPTION OF THE DRAWINGS

FIG. 1 is a front and rear view of a garment for a user of a medical device to be worn on a lower body portion, consistent with the present inventive concepts;

FIG. 1A is a perspective view of a fastener described in FIG. 1, consistent with the present inventive concepts;

FIG. 2 is a front and rear view of a garment for a user of a medical device to be worn on a lower body portion, including the medical device, consistent with the present inventive concepts;

FIG. 3 is a front and rear view of a garment for a user of a medical device to be worn on an upper body portion, consistent with the present inventive concepts; and

FIG. 4 is a front view of a garment for a user of a medical device to be worn as a brassiere; consistent with the present inventive concepts.

### DETAILED DESCRIPTION OF THE DRAWINGS

Reference will now be made in detail to the present embodiments of the technology, examples of which are illustrated in the accompanying drawings. The same reference numbers are used throughout the drawings to refer to the same or like parts.

The terminology used herein is for the purpose of describing particular embodiments and is not intended to be limiting of the inventive concepts. As used herein, the singular forms “a,” “an” and “the” are intended to include the plural forms as well, unless the context clearly indicates otherwise.

It will be further understood that the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) when used herein, specify the presence of stated features, integers, steps, operations, elements, and/or components, but do not preclude the presence or addition of one or more other features, integers, steps, operations, elements, components, and/or groups thereof.

It will be understood that, although the terms first, second, third, etc. may be used herein to describe various limitations, elements, components, regions, layers and/or sections, these limitations, elements, components, regions, layers and/or sections should not be limited by these terms. These terms are only used to distinguish one limitation, element, component, region, layer or section from another limitation, element, component, region, layer or section. Thus, a first limitation, element, component, region, layer or section discussed below could be termed a second limitation, element, component, region, layer or section without departing from the teachings of the present application.

It will be further understood that when an element is referred to as being “on”, “attached”, “connected” or “coupled” to another element, it can be directly on or above, or connected or coupled to, the other element or intervening elements can be present. In contrast, when an element is referred to as being “directly on”, “directly attached”, “directly connected” or “directly coupled” to another element, there are no intervening elements present. Other words used to describe the relationship between elements should be interpreted in a like fashion (e.g., “between” versus “directly between,” “adjacent” versus “directly adjacent,” etc.).

Spatially relative terms, such as “beneath,” “below,” “lower,” “above,” “upper” and the like may be used to describe an element and/or feature's relationship to another element(s) and/or feature(s) as, for example, illustrated in the figures. It will be understood that the spatially relative terms are intended to encompass different orientations of the device in use and/or operation in addition to the orientation depicted in the figures. For example, if the device in a figure is turned over, elements described as “below” and/or “beneath” other elements or features would then be oriented “above” the other elements or features. The device can be otherwise oriented (e.g., rotated 90 degrees or at other orientations) and the spatially relative descriptors used herein interpreted accordingly.

The term “and/or” where used herein is to be taken as specific disclosure of each of the two specified features or components with or without the other. For example “A and/or B” is to be taken as specific disclosure of each of (i) A, (ii) B and (iii) A and B, just as if each is set out individually herein.

It is appreciated that certain features of the invention, which are, for clarity, described in the context of separate embodiments, may also be provided in combination in a single embodiment. Conversely, various features of the invention which are, for brevity, described in the context of a single embodiment, may also be provided separately or in any suitable sub-combination.

For example, it will be appreciated that all features set out in any of the claims (whether independent or dependent) can be combined in any given way.

The term “garment” as used herein can include any article of clothing for males and/or females, including but not limited to an undergarment such as underwear, a pair of boxers, a pair of boxer-briefs, a thong, or a pair of boy-shorts (type of undergarment typically worn by a female); a shirt such a long-sleeved shirt, a short-sleeved shirt, a camisole, or a sleeveless shirt; a pair of shorts; a pair of pants; swimwear; athletic wear; a brassiere; a sports brassiere; a belt; and the like.

Referring now to FIG. 1, a front and rear view of a garment for a user of a medical device to be worn on a lower body portion is illustrated. Garment 10 comprises front panel 20, back panel 30, and a pair of leg openings 50a and 50b. In the illustrated embodiment, garment 10 can be worn on the lower body portion of a user of a medical device, for example garment 10 can comprise an undergarment, a pair of shorts, swimwear, or the like. Garment 10 comprises at least one pocket 60 configured to support a portion of a medical device, for example pockets 60a and 60b. Garment 10 further comprises waistband 40 where a tubular structure such as a tube, a catheter, a wire, a cable, a cord, or the like of a medical device can be secured to a surface (inner and/or outer surface) of waistband 40, for example via at least one fastener 70, for example fasteners 70a and 70b.

Garment 10 can be worn by a user of a medical device, including but not limited to, an insulin pump; an infusion device; a blood sugar monitoring device; an insulin inhaler; or a debifrillator. For example, where the medical device comprises an insulin pump device, the pump portion, including controls, a processing module, batteries and an insulin reservoir can be supported or stored in pocket 60a or 60b.

Pocket 60 can be sized to accommodate any medical device. In some embodiments, pocket 60 dimensions are approximately 77 mm (length)×47 mm (width)×18 mm (height). In some embodiments, pocket 60 dimensions are approximately 80 mm (length)×51 mm (width)×24 mm (height). In some embodiments, pocket 60 dimensions are approximately 80 mm (length)×47 mm (width)×24 mm (height). In some embodiments, pocket 60 dimensions are approximately 76.2 mm (length)×50.8 mm (width)×20.3 mm (height). In some embodiments, pocket 60 dimensions are approximately 77 mm (length)×51 mm (width)×18 mm (height). Pocket 60 can be configured to support up to approximately 16 ounces, for example a medical device component weighing approximately 3 ounces to 5 ounces.

Pocket 60 can be open or closed. For example, pocket 60 can be closed via a hook and loop fastener, e.g. Velcro®; one or more buttons; a zipper; and combinations thereof.

Pocket 60 can comprise the same, similar or different material as front panel 20, back panel 30 and/or any other pocket included on garment 10.

Any number of pockets 60 can be used and positioned on any portion of front panel 20 and/or back panel 30. In embodiments where garment 10 includes more than one pocket, each pocket can be of similar or dissimilar construction, e.g. size, material, open or closed, and the like. In the embodiment illustrated in FIG. 1, garment 10 includes two pockets 60a and 60b, where both pockets 60a and 60b are positioned on front panel 20. In some embodiments, pockets 60a and 60b can be positioned on each hip region. In some embodiments, pockets 60a and 60b can be located on back panel 30. It may be desirable to have at least two pockets so that a user can select a preferred position of the medical device components. For example, in the case where the medical device is an insulin pump, the user may change the injection site periodically, therefore the ability to select the location for the pump component of the insulin pump maybe desirable. The pocket not occupied by the pump component of the insulin pump can be used for various purposes, for example to store additional insulin supplies such an extra bottle of insulin, a needle, a catheter, a battery, tubing, a source of glucose, an emergency glucose treatment, and the like.

Waistband 40 can comprise a same, similar, or different material than front panel 20, back panel 30, and/or one or more pockets 60a and/or 60b. For example, in an embodiment where garment 10 is an undergarment, waistband 40 can comprise an elastic material. In some embodiments, the tubular structure has a length of at least approximately 20″ long, for example a length of approximately 25″ long and can be positioned along a surface of waistband 40 and secured in place by one or more fasteners 70, for example fasteners 70a and 70b. Similarly, the tubular structure can be placed secured to a surface of front panel 20 and/or back panel 30 defining leg opening 50a or 50b via one or more fasteners 70.

Fasteners 70a and 70b are configured to secure the tubular structure to a surface of waistband 40, for example the inner and/or outer surface of waistband 40. Fastener 70 can be provided as a separate component or can be integral to waistband 40, e.g. sewn in or otherwise fixed to waistband 40. Combinations of integral fasteners 70 and separate component fasteners 70 can be provided, for example, two fasteners 70, such as fasteners 70a and 70b can be sewn into garment 10, and additionally can be provided for optional use. Fastener 70 can be configured so as to limit or avoid pinching, kinking, or otherwise affecting the functionality of a tubular structure. For example, fastener 70 can comprise a bobby-pin-like construction as shown in FIG. 1A, having closed end 72 and opening 71 at the opposite end. Continuing with this example, the tubular structure can rest in opening 71, while being secured in place by closed end 72. In some embodiments, opening 71 can be sized to allow a tubular structure having an outer diameter ranging from approximately 0.1 mm to 10 mm to pass therethrough.

In the illustrated embodiment, garment 10 comprises two fasteners 70a and 70b, both positioned on waistband 40 (front panel 20 portion of waistband 40). However, garment 10 can include any number of fasteners 70 placed in any position about waistband 40. In some embodiments, five fasteners 70 can be spaced evenly on waistband 40. In some embodiments, five fasteners 70 can be utilized, where two are positioned on front panel 20, two are positioned on each hip region, and one is positioned in the middle of back panel 30. The number of fasteners 70 used can be at the option of the user, for example, where garment 10 is provided with multiple fasteners 70, and the user can employ as many fasteners 70 as desired or necessary.

Other examples of fasteners 70 include but are not limited to, a clip; a pin; a medical hose having a reinforced wire; a loop such as fabric loop similar to a belt-loop (similar to fastener 80 of FIGS. 3 and 4 below); and combinations thereof. Fastener 70 can be constructed of any suitable material, for example, metal; plastic; fabric; rubber; elastic material; and combinations thereof.

In some embodiments, waistband 40 can include a pouch, not shown but similar to the space for a drawstring for a pair of sweatpants, configured to house a tubular structure. In these embodiments, waistband 40 can further include at least one opening, for example a first opening and a second opening. For example, the first opening can be configured for insertion and removal of the tubular structure into the pouch that travels around the circumference of waistband 40, and the second opening can be configured for access to the injection site, meaning the needle end of the tubular structure can exit the pouch at the second opening and be injected into skin. A third opening can be included to assist in feeding the tubular structure through the pouch. In some embodiments, the pouch can be formed in another portion of garment 10, for example, proximate leg openings 50a and/or 50b such as within a seam around a circumference of leg opening 50a or 50b.

Garment 10 can be made of various materials, non-limiting examples include cotton; lyocell; bamboo; viscose; elastane; polyester; nylon; polychloroprene; and combinations thereof. In some embodiments, for example when garment 10 is swimwear or a component of athletic wear, garment 10 can be waterproof.

As described above, garment 10 can be worn by a user of a medical device, including but not limited to, an insulin pump; an infusion device; a blood sugar monitoring device; insulin inhaler; or a defibrillator. Additionally, garment 10 can be used to store any items that may be useful to one having a particular disease or disorder. For example, a diabetic may store a supplemental insulin reservoir in pocket 60. Another example is an emergency glucose treatment that can be stored in pocket 60 to be used if a hypoglycemic reaction occurs.

Referring now to FIG. 2, a front and rear view of a garment for a user of a medical device to be worn on a lower body portion, including the medical device is illustrated. Garment 10 is of the same or similar construction as garment 10 described in FIG. 1, including front panel 20, back panel 30, and a pair of leg openings 50a and 50b. Garment 10 comprises pocket 60a positioned on a hip region and pocket 60b positioned on the opposite hip region. In the illustrated embodiment, pump portion 120, including controls, a processing module, batteries and an insulin reservoir, of an insulin pump is stored in pocket 60b, however can be stored in either pocket 60a or 60b at the option of the user. Garment 10 further comprises waistband 40 where tubing 110 can be secured to the outer surface of waistband 40, for example via at least one fastener 80.

In the illustrated embodiment, fasteners 80 comprise a loop configuration, similar to a belt loop on pair of shorts or pants, for example. One or more fasteners 80 can be integral to garment 10′, for example sewn in position on the outer surface of waistband as shown. Alternatively or additionally, fasteners 80 can be positioned on an inner surface of waistband 40. In some embodiments, garment 10 can include fasteners similar to fasteners 70 described above.

In some embodiments, pocket 60 is sized to accommodate pump 120 having dimensions of approximately 80 mm (length)×51 mm (width)×24 mm (height) and weighing approximately 3 ounces to 5 ounces.

As an example, and in no particular order, a user of an insulin pump can put on garment 10; inject the needle portion of the device, including a female connector, at the desired location along the user's torso or buttocks; place pump portion 120 into pocket 60a or 60b; secure tubing 110 to a surface of waistband 40 via one or more fasteners 80; and connect tubing 110 to the female connector.

Referring now to FIG. 3, a front and rear view of a garment for a user of a medical device to be worn on an upper body portion is illustrated. Garment 10′ comprises front panel 20, back panel 30, and a pair of arm openings 51a and 51b. In the illustrated embodiment, garment 10′ can be worn on an upper body portion by a user of a medical device, for example garment 10′ can comprise a short-sleeved shirt, a long-sleeved shirt, a camisole, or a sleeveless shirt. Garment 10′ comprises at least one pocket 60 configured to support a portion of a medical device, for example pockets 60a and 60b. A tubular structure of a medical device can be secured to a surface (inner and/or outer surface) of front panel 20 and/or back panel 30, for example via at least one fastener 80.

In the illustrated embodiment, garment 10′ includes pocket 60a positioned on front panel 20 and pocket 60b positioned on back panel 30. However, pockets 60a and 60b can be positioned in any location on front panel 20 and/or back panel 30, and any number of pockets can be included.

In the illustrated embodiment, fasteners 80 comprise a similar configuration to fastener 80 described in FIG. 2. In some embodiments, garment 10 can include fasteners similar to fasteners 70 described above.

All components of garment 10′, including front panel 20, back panel 30, and the pockets 60a and 60b, can be the same or similar construction and configuration as described in FIG. 1 and support a medical device as described in FIG. 2 above.

Referring now to FIG. 4, a front view of a garment for a user of a medical device to be worn as a brassiere is illustrated. Garment 10″ comprises front panel 20, back panel 30, and a pair of arm openings 51a and 51b. In the illustrated embodiment, garment 10″ can be worn as a brassiere or a sports brassiere by the user of a medical device. Garment 10″ comprises at least one pocket 60 configured to support a portion of a medical device, for example pocket 60a and 60b. A tubular structure of a medical device can be fastened to a surface (inner and/or outer surface) of front panel 20 and/or back panel 30, for example via at least one fastener 80. Fasteners 80 can be configured similar to fasteners 80 of FIG. 3 and/or garment can include fasteners similar to fasteners 70 of FIG. 1.

In the illustrated embodiment, garment 10″ includes both pockets 60a and 60b positioned on back panel 30. However, pockets 60a and 60b can be positioned in any location on front panel 20 and/or back panel 30, and any number of pockets can be included. In some embodiments, garment 10″ includes a pocket 60 located in mid portion of front panel 20.

All components of garment 10″, including front panel 20, back panel 30, at least one pocket 60, can be of similar construction and configuration as described in FIG. 1 and support a medical device as described in FIG. 2 above.

While specific embodiments of a garment have been described, any garment can be configured for a user of a medical device consistent with the concepts described herein.

While the embodiments have been described in reference to the environment in which they were developed, they are merely illustrative of the principles of the inventions. Modification or combinations of the above-described embodiments, other embodiments, configurations, and methods for carrying out the invention, and variations of aspects of the invention that are obvious to those of skill in the art are intended to be within the scope of the claims. In addition, where this application has listed the steps of a method or procedure in a specific order, it may be possible, or even expedient in certain circumstances, to change the order in which some steps are performed, and it is intended that the particular steps of the method or procedure claim set forth herebelow not be construed as being order-specific unless such order specificity is expressly stated in the claim.

I claim:

1. A garment for a medical device comprising:
a front panel;
a back panel;
at least one opening for a body part;
at least one pocket; and
at least one fastener.

2. The garment of claim 1 wherein the garment comprises a garment to be worn by a male or a female user of a medical device selected from the group consisting of: an undergarment such as underwear, a pair of boxers, a pair of boxer-briefs, a thong, or a pair of boy-shorts; a shirt such a long-sleeved, a short-sleeved, or a sleeveless shirt; a pair of shorts; a pair of pants; swimwear; athletic wear; a camisole; a brassiere; a sports brassiere; and a belt.

3. The garment of claim 1 wherein the garment comprises a material selected from the group consisting of: cotton; lyocell; bamboo; viscose; elastane; polyester; nylon; polychloroprene; a waterproof material; and combinations thereof.

4. The garment of claim 1 further comprising a waistband and wherein the at least one opening comprises a pair of leg openings.

5. The garment of claim 4 wherein the medical device comprises a tubular structure selected from the group consisting of: a tube; a catheter; a wire; a cable; a cord; and combinations thereof, and wherein the tubular structure is secured to a surface of the waistband via the at least one fastener.

6. The garment of claim 5 comprising at least five fasteners, wherein two of the at least five fasteners are positioned on the front panel, one of the at least five fasteners is positioned on the back panel, one of the at least five fasteners is positioned on a hip region, and one of the at least five fasteners is positioned on an opposite hip region.

7. The garment of claim 1 wherein the at least one opening comprises a pair of arm openings.

8. The garment of claim 7 wherein the medical device comprises a tubular structure selected from the group consisting of: a tube; a catheter; a wire; a cable; a cord; and combinations thereof, and wherein the tubular structure is secured to a surface of the front panel and/or a surface of the back panel.

9. The garment of claim 1 wherein the at least one pocket is configured to support a portion of the medical device.

10. The garment of claim 1 wherein the portion comprises a pump portion of an insulin pump.

11. The garment of claim 1 wherein the at least one pocket is sized to support a portion of the medical device having dimensions of approximately 77 mm (length)×47 mm (width)×18 mm (height).

12. The garment of claim 1 wherein the at least one pocket comprises a material having minimal stretching.

13. The garment of claim 1 wherein the at least one pocket is positioned on the front panel and/or the back panel.

14. The garment of claim 1 wherein the at least one pocket comprises a first pocket and a second pocket wherein the first pocket is positioned on a hip region and the second pocket is positioned on the opposite hip region.

15. The garment of claim 1 wherein the at least one pocket is configured to support up to approximately 16 ounces.

16. The garment of claim 1 wherein the at least one pocket comprises a hook and loop fastener; at least one button; a zipper; and combinations thereof configured to close the at least one pocket.

17. The garment of claim 1 wherein the at least one fastener is configured to secure a tubular structure of the medical device to the garment.

18. The garment of claim 16 wherein the at least one fastener is integral to the garment.

19. The garment of claim 16 wherein the at least one fastener is a separate component.

20. The garment of claim 18 wherein the at least one fastener comprises a first end and a second end, and wherein the first end comprises a closed portion and the second end comprises an opening configured to limit pinching, kinking or affecting the functionality of the tubular structure.

21. The garment of claim 19 wherein the opening is sized to allow a tubular structure having an outer diameter ranging from approximately 0.1 mm to 10 mm to pass therethrough.

22. The garment of claim 16 wherein the at least one fastener comprises a fastener selected from the group consisting of: a pin; a clip; a medical hose with a reinforcing wire; a loop; and combinations thereof.

23. The garment of claim 1 wherein the medical device comprises an insulin pump; an infusion device; a blood sugar monitoring device; an insulin inhaler; or a defibrillator.

24. A garment for a medical device comprising:
a front panel;
a back panel;
a pair of leg openings;
a waistband;
at least one pocket configured to support a portion of the medical device; and
at least one fastener configured to secure a tubular structure of the medical device to a surface of the waistband.

25. A garment for a medical device comprising:
a front panel;
a back panel;
a pair of arm openings;
at least one pocket configured to support a portion of the medical device; and
at least one fastener configured to support a tubular structure of the medical device to a surface of the front panel and/or a surface of the back panel.
